SEQUENCE LISTING (22 sequences)

SEQ ID NO: 1 - DNA, 276 bp, Mus musculus
  source 1..276: mol_type = other DNA
atgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagctaa

SEQ ID NO: 2 - protein, 91 aa, Mus musculus
MKISAAALTIILTAAALCTPAPASPYGSDTTPCCFAYLSLALPRAHVKEYFYTSSKCSNLAVVFVTRRNRQVCANPEKKWVQEYINYLEMS

SEQ ID NO: 3 - RNA, 276 nt, Mus musculus
  source 1..276: mol_type = mRNA
atgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagctaa

SEQ ID NO: 4 - DNA, 276 bp, Homo sapiens
  source 1..276: mol_type = other DNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 5 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 6 - RNA, 276 nt, Homo sapiens
  source 1..276: mol_type = mRNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 7 - DNA, 22 bp, synthetic construct
  source 1..22: mol_type = other DNA
gtgccaacccagagaagaagtg

SEQ ID NO: 8 - DNA, 20 bp, synthetic construct
  source 1..20: mol_type = other DNA
atgcccattttcccaggacc

SEQ ID NO: 9 - DNA, 23 bp, synthetic construct
  source 1..23: mol_type = other DNA
tgtcgttgttgtatagggagttc

SEQ ID NO: 10 - DNA, 20 bp, synthetic construct
  source 1..20: mol_type = other DNA
agcatgtgtctggaaggatg

SEQ ID NO: 11 - DNA, 18 bp, synthetic construct
  source 1..18: mol_type = other DNA
caagctgagctggctctc

SEQ ID NO: 12 - DNA, 20 bp, synthetic construct
  source 1..20: mol_type = other DNA
acgactgactaaagcactgg

SEQ ID NO: 13 - DNA, 22 bp, synthetic construct
  source 1..22: mol_type = other DNA
ggatcaccttctgcttccttac

SEQ ID NO: 14 - DNA, 22 bp, synthetic construct
  source 1..22: mol_type = other DNA
gcaactagagacatgggtgatg

SEQ ID NO: 15 - DNA, 21 bp, synthetic construct
  source 1..21: mol_type = other DNA
cttctcaccttcaccacacag

SEQ ID NO: 16 - DNA, 19 bp, synthetic construct
  source 1..19: mol_type = other DNA
tgtgacccaagcacagttc

SEQ ID NO: 17 - DNA, 19 bp, synthetic construct
  source 1..19: mol_type = other DNA
cagggtagcagaggaagtg

SEQ ID NO: 18 - DNA, 22 bp, synthetic construct
  source 1..22: mol_type = other DNA
gcagttagaggcagagtcatac

SEQ ID NO: 19 - DNA, 21 bp, synthetic construct
  source 1..21: mol_type = other DNA
cttctcaccttcaccacacag

SEQ ID NO: 20 - DNA, 19 bp, synthetic construct
  source 1..19: mol_type = other DNA
tgtgacccaagcacagttc

SEQ ID NO: 21 - DNA, 21 bp, synthetic construct
  source 1..21: mol_type = other DNA
tgctaccctggctccctataa

SEQ ID NO: 22 - DNA, 22 bp, synthetic construct
  source 1..22: mol_type = other DNA
gatgcatgtgctgtctcagagt

(19) United States
(12) Patent Application Publication
(10) Pub. No.: US 2025/0188140 A1
(43) Pub. Date: Jun. 12, 2025

(54) CCL5 MRNA NANOPARTICLE AND METHODS OF USE THEREOF

(71) Applicant: Ohio State Innovation Foundation, Columbus, OH (US)

(72) Inventors: Xue-Feng BAI, Columbus, OH (US); Yizhou DONG, New York, NY (US); Chunxi ZENG, Chelmsford, MA (US); Jin-Qing LIU, Columbus, OH (US)

(21) Appl. No.: 18/844,596

(22) PCT Filed: Mar. 8, 2023
(86) PCT No.: PCT/US2023/063922

§ 371 Date: Sep. 6, 2024

Related U.S. Application Data
(60) Provisional application No. 63/345,171, filed on May 24, 2022.
(60) Provisional application No. 63/317,723, filed on Mar. 8, 2022.

(51) Int. Cl. C07K 14/52; A61K 9/51; A61P 35/00; C12N 15/88

(52) U.S. Cl. CPC C07K 14/523; A61K 9/5123; A61P 35/00; C12N 15/88

(57) ABSTRACT

The present disclosure relates to a lipid-like nanoparticle encapsulating a C—C motif chemokine ligand 5 (CCL5) mRNA and methods of use thereof.

### CROSS-REFERENCE TO RELATED APPLICATIONS

The application claims the benefit of U.S. Provisional Application No. 63/317,723, filed Mar. 8, 2022, and U.S. Provisional Application No. 63/345,171, filed May 24, 2022, which are hereby incorporated herein by reference in their entirety.

### STATEMENT REGARDING FEDERALLY SPONSORED RESEARCH

This invention was made with Government Support under Grant No. R01 CA229254 awarded by the National Institutes of Health. The Government has certain rights in the invention.

### FIELD

The present disclosure relates to a lipid-like nanoparticle encapsulating a C—C motif chemokine ligand 5 (CCL5) mRNA and methods of use thereof.

### BACKGROUND

IL-27 is a member of the IL-12 cytokine family that consists of an IL-12 p40-related protein subunit, EBV-induced gene 3 (EBI3), and a p35-related subunit, p28. IL-27 signals through a heterodimeric receptor (IL-27R) composed of the WSX-1 (IL-27Rα) and the gp130 subunits in a variety of cell types, including T lymphocytes. IL-27R signaling enhances the recruitment of several JAK family kinases and activates STAT family transcription factors 1 and 3. IL-27 enhances Th1/Tc1 responses by activating the Stat1-T-bet axis and promotes T cell expression of T-bet, Eomes, IL-12Rβ2, granzyme B and Perforin. It also inhibits Th2 and Th17 responses by blocking the expression of transcription factors GATA-3 (Th2) and RoRγτ (Th17) and is an inducer of IL-10 production by T cells. Moreover, IL-27 can also induce PD-L1 expression in T cells which restrain T cell effector functions by interacting with PD-1 on T cells. These functional properties of IL-27 show that it enhances anti-tumor immunity and inhibits Th17/Th2 mediated autoimmunity.

Therefore, there is a need to address development of IL-27 therapeutic strategies and other shortcomings associated with treating and preventing tumor microenvironments surrounding cancerous tissues.

The compounds, compositions, and methods disclosed herein address these and other needs.

### SUMMARY

The present disclosure provides lipid-like nanoparticles encapsulating a CCL5 mRNA and uses thereof to treat cancer. Also provided are compositions of the lipid-like nanoparticles and sequences of the CCL5 mRNA. The present disclosure also provides methods using the compositions for administering the treatment to a subject.

In one aspect, disclosed herein a composition comprising a lipid-like nanoparticle encapsulating a CCL5 mRNA. In some embodiments, the CCL5 mRNA is encoded by SEQ ID NO: 1 or SEQ ID NO: 4. In some embodiments, the CCL5 mRNA encodes a protein comprising SEQ ID NO: 2 or SEQ ID NO: 5. In some embodiments, the CCL5 mRNA comprises SEQ ID NO: 3 or SEQ ID NO: 6.

In one embodiment, the composition comprises a compound of Formula I:

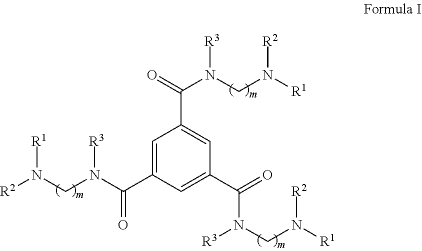

and salts thereof, wherein R¹, R², R³, and m are described herein.

In some embodiments, the composition comprises a compound of Formula II:

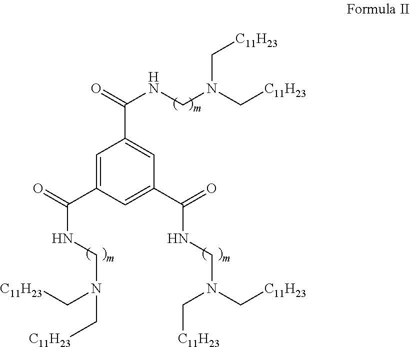

and salts thereof.

In some embodiments, the lipid-like nanoparticle comprises a non-cationic lipid, a polyethylene glycol-lipid, and a sterol. In one embodiment, the non-cationic lipid is a phosphatidylethanolamine lipid. In some embodiments, the phosphatidylethanolamine lipid is selected from 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), or combinations thereof. In some embodiments, the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs. In some embodiments, the sterol is selected from cholesterol, campesterol, ergosterol, or sitosterol.

In one aspect, disclosed herein is a method of treating a cancer in a subject comprising administering a composition comprising a lipid-like nanoparticle encapsulating a CCL5 mRNA.

In some embodiments, the CCL5 mRNA is encoded by SEQ ID NO: 1. In some embodiments, the CCL5 mRNA encodes a protein comprising SEQ ID NO: 2. In some embodiments, the subject is further administered an anticancer agent. In some embodiments, the anticancer agent is an immunotherapeutic agent. In some embodiments, the anticancer agent is selected from a PD-L1 antibody, a PD-1 antibody, or a CTLA-4 antibody. In some embodiments, the anticancer agent is a CTLA-4 antibody.

In some embodiments, the lipid-like nanoparticle comprises a compound of Formula I:

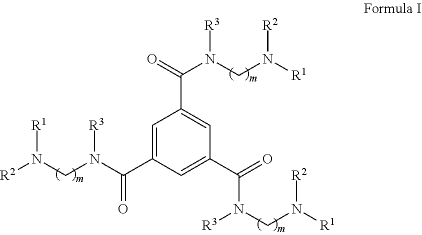

and salt thereof, wherein R¹, R², R³, and m are described herein.

In some embodiments, the lipid-like nanoparticle comprises a compound of Formula II:

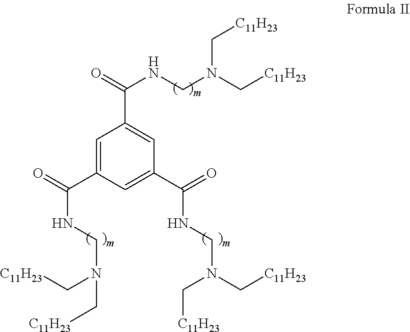

and salts thereof.

In some embodiments, the lipid-like nanoparticle further comprises a non-cationic lipid, a polyethylene glycol-lipid, and a sterol. In one embodiment, the non-cationic lipid is a phosphatidylethanolamine lipid. In some embodiments, the phosphatidylethanolamine lipid is selected from 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), or combinations thereof. In some embodiments, the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs. In some embodiments, the sterol is selected from cholesterol, campesterol, ergosterol, or sitosterol.

In some embodiments, the cancer is a melanoma.

### BRIEF DESCRIPTION OF THE DRAWINGS

The accompanying figures, which are incorporated in and constitute a part of this specification, illustrate several aspects described below.

FIGS. 1A-1D show IL-27 induces CCL5 production by mouse and human T cells in vitro. FIG. 1A shows C57BL6 mice treated with AAV-IL-27 or AAV-ctrl virus (2×10¹¹ DRP/mouse i.m.). Three weeks after AAV injection, mice were sacrificed, CD8⁺ T cells were purified from AAV-treated mice and submitted for RNAseq analysis. Major differentially expressed genes are shown. FIG. 1B shows naïve CD4 or CD8 T cells were purified from spleen and lymph nodes from C57BL6 mice. Cells were activated with anti-CD3/CD28 beads with or without recombinant mouse IL-27 (50 ng/ml). Production of CCL5 was detected by flow cytometry on day 3 or day 5 after T cell culture. FIG. 1C shows spleen cells from P1CTL TCR transgenic mice were activated with 0.1 ug/ml of P1A35-43 with or without recombinant IL-27 (50 ng/ml). Production of CCL5 by CD8⁺Vα8.3⁺ cells was detected by flow cytometry on day 3 or day 5 after T cell culture. FIG. 1D shows CD8⁺ T cells were purified from human PBMC. Cells were activated with anti-CD3/CD28 beads with or without recombinant hIL-27 (50 ng/ml). Production of CCL5 was detected by flow cytometry on day 3 or day 5 after T cell culture. Data are expressed as Mean±SD of 3-6 samples in each group, and data represent 3-5 experiments with similar results *P<0.01, **P<0.001, ***P<0.0001 by student's t-test.

FIGS. 2A-2B shows IL-27 induction by CCL5 production in T cells is IL-27R-dependent. FIG. 2A shows IL-27 induction in naïve CD4⁺. FIG. 2B shows IL-27 induction in naïve CD8⁺ T cells. Naïve CD4⁺ and CD8⁺ T cells were purified from spleen and lymph nodes from CD45.1⁺ C57BL6 mice or CD45.2⁺ IL-27Rα^{-/-} mice. Cells were activated with anti-CD3/CD28 beads with or without recombinant mouse IL-27 (50 ng/ml) separately or in mixture (1:1 ratio). Production of CCL5 was detected by flow cytometry on day 5 after T cell culture. Data are expressed as Mean±SD of 3 samples in each group, and data represent three experiments with similar results. *P<0.01, **P<0.001, ***P<0.0001 by student's t-test.

FIGS. 3A-3B shows IL-27 induced CCL5 production by T cells in the absence of Stat1. Naïve CD4 or CD8 T cells were purified from spleen and lymph nodes from Stat1^{-/-} and control BALB/c mice. Cells were activated with anti-CD3/CD28 beads with or without recombinant mouse IL-27 (50 ng/ml). FIG. 3A shows the production of CCL5 in CD4⁺ T cells. FIG. 3B shows the production of CCL5 in CD8⁺ T cells. Production of CCL5 in CD4⁺ and CD8⁺ T cells were detected by flow cytometry on day 3 or day 5 after T cell culture. Data are expressed as Mean±SD of 3 samples in each group, and data represent four experiments with similar results. **P<0.001, ***P<0.0001 by student's t-test.

FIGS. 4A-4D shows the ChIP assay of Stat1 and Stat3 binding in CCL5 promotor. P1CTL TCR transgenic T cells were activated with 0.1 ug/ml of P1A35-43 with or without recombinant IL-27 (50 ng/ml). FIG. 4A shows the relative expression of CCL5 mRNA were determined by RT-qPCR. Data are expressed as Mean±SD of 3 samples in each group, and data represent two experiments with similar results. FIG. 4B shows the binding of RNA polymerase II to CCL5 promoter was confirmed by ChIP using an anti-RNA pol II antibody. FIG. 4C shows the ChIP assay was performed on P1CTL cells using qPCR primers designed to probe the two putative binding sties of Stat3. Both the −200 bp and −4700 bp sites upstream of CCL5 TSS revealed significant binding of STAT3. *P<0.05 by student's t test. FIG. 4D shows the ChIP assay was performed using qPCR primers designed to probe the binding sites of Stat1 in CCL5 promotor. Moderate binding of Stat1 to the −4700 bp site but not to the other three putative sites were detected. *P<0.05 by student's t-test.

FIGS. 5A-5B shows AAV-IL-27 treatment upregulates CCL5 in T cells in vivo. FIG. 5A shows that AAV-IL-27 or AAV-ctrl virus were injected into IL-27Rα^{-/-} and control C57BL6. FIG. 5B shows that AAV-IL-27 or AAV-ctrl virus were injected into Stat1^{-/-}BALB/c and control BALB/c mice. All mice were injected intramuscularly (i.m.) at a dose of 2×10¹¹ DRP. Three weeks later, mice were sacrificed and T cell production of CCL5 in spleens were analyzed by flow cytometry. Data shown represent 3-5 experiments with similar results. ns: no significant difference. **P<0.01, ***P<0.001 by student's t-test.

FIGS. 6A-6B shows that AAV-IL-27 therapy induces CCL5 production in tumor-infiltrating T cells. FIG. 6A shows that C57BL6 mice were first treated with AAV-IL-27 or AAV-ctrl virus (2×10¹¹ DRP/mouse i.m.) Mice were also challenged with B16. F10 tumor cells (1×10⁵ cells/mouse) s.c. Three weeks after AAV injection, T cell expression of CCL5 was evaluated by flow cytometry. *P<0.05, **P<0.01, ***P<0.001 by student's t test. FIG. 6B shows that IL-27Rα^{-/-} and control C57BL6 mice were first treated with AAV-IL-27 or AAV-ctrl virus (2×10¹¹ DRP/mouse i.m.). Mice were also challenged with B16.F10 tumor cells (1×10⁵ cells/mouse) s.c. Three weeks after AAV injection, T cell expression of CCL5 in tumors was evaluated by flow cytometry. *P<0.05, **P<0.01, ***P<0.001 by student's t test. Data shown represent three experiments with similar results.

FIGS. 7A-7C shows that IL-27-induced CCL5 production contributes to anti-tumor immunity. FIG. 7A shows C57BL6 mice were inoculated with B16.F10 melanoma cells (1×10⁵ cells/mouse) s.c. Mice were treated with AAV-IL-27 or AAV-ctrl virus (2×10¹¹ DRP/mouse i.m.) four days later. On day 13, 17, 22 and 26 mice were also treated with anti-CCL5 (100 μg/mouse) or a control IgG2a antibody (100 μg/mouse) i.p. Tumor growth was monitored, and tumor volume was calculated. Five to seven mice were used for the experiment and data represents two experiments with similar results. *P<0.05, **P<0.01 by one way ANOVA. FIG. 7B shows that B16.F10 cells were co-cultured with control NP or NP-CCL5 mRNA (50 ng mRNA/ml). Eighteen hours later, the supernatants of cell cultures were collected, and ELISA was used to measure CCL5 concentrations in culture supernatants. FIG. 7C shows that C57BL6 mice were inoculated with B16.F10 melanoma cells (1×10⁵ cells/mouse) subcutaneously (s.c.). When tumors were fully established on day 10, mice received intratumor injection of either NP-CCL5 mRNA (2 μg/mouse; n=7) or vehicle control (n=6) every other day for 6 times. Tumor size was measured every two days and calculated as tumor volume. ***P<0.001; ****P<0.0001 by one way ANOVA.

FIG. 8. Analysis of ChIP-seq data deposited to Gene Expression Omnibus (Accession Number GSE65621) reveals the putative binding sites of STAT1 and STAT3 upstream of CCL5 transcription starting site (TSS).

FIG. 9. AAV-IL-27 therapy failed to induce CCL5 in non-T lymphoid cells. C57BL6 mice were treated with AAV-IL-27 or AAV-ctrl virus (2×10¹¹ DRP/mouse i.m.). Three weeks after AAV injection, CCL5 expression in non-T immune cells in spleens was evaluated by flow cytometry. Data shown represent three experiments with similar results.

### DETAILED DESCRIPTION

The present disclosure provides lipid-like nanoparticles encapsulating a CCL5 mRNA and uses thereof to treat cancer. Also provided are compositions of the lipid-like nanoparticles and sequences of the CCL5 mRNA. The present disclosure also provides methods using the compositions for administering the treatment to a subject.

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “treat,” “treating.” “treatment.” “therapy”, and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments may be applied preventively, prophylactically, palliatively or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The term “cancer” is used to address any neoplastic disease. It includes both solid tumors and hematologic malignancies, including, for example, epithelial (surface and glandular) cancers, soft tissue and bone sarcomas, angiomas, mesothelioma, melanoma, lymphomas, leukemias and myeloma.

As used herein, the term “lipid” or “lipid-like” refers to a macromolecule that is soluble in nonpolar solvents. These molecules are usually hydrophobic or amphiphilic molecules; the amphiphilic nature of some lipids allows formation of structures such as vesicles, liposomes, membranes, and nanoparticles.

As used herein, the term “encapsulate” or “encapsulating” refers to a process in which molecules, such as nucleic acids, proteins, and other macromolecules are surrounded or coated by nanoparticles for delivery to a targeted tissue or cell-type.

As used herein, the term “chemical compound” and “compound”, refers to a chemical substance consisting of two or more different types of atoms or chemical elements in a fixed stoichiometric proportion. These compounds have a unique and defined chemical structure held together in a defined spatial arrangement by chemical bonds. Chemical compounds can be held together by covalent bonds, ionic bonds, metallic ions, or coordinate covalent bonds.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences) A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues, always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or jodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “phosphonyl” is used herein to refer to the phospho-oxo group represented by the formula —P(O)(OZ¹)₂, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

Compositions and Compounds The present disclosure provides lipid-like nanoparticles encapsulating a CCL5 mRNA and uses thereof to treat cancer. The present disclosure also provides methods using the compositions for administering the treatment to a subject.

In one aspect, disclosed herein is a composition comprising a lipid-like nanoparticle encapsulating a CCL5 mRNA. In some embodiments, the composition comprises a lipid-like nanoparticle encapsulating a CCL5 peptide. In some embodiments, the CCL5 mRNA is encoded by SEQ ID NO: 1. In some embodiments, the CCL5 mRNA encodes a protein comprising SEQ ID NO: 2.

In some embodiments, the CCL5 mRNA is encoded by a sequence at least 60% (for example, at least 60%, at least 65%, at least 70%, at least 75%, at least 80%, at least 85%, at least 90%, at least 95%, or more) identical to SEQ ID NO: 1. In some embodiments, the CCL5 mRNA encodes a protein sequence at least 60% (for example, at least 60%, at least 65%, at least 70%, at least 75%, at least 80%, at least 85%, at least 90%, at least 95%, or more) identical to SEQ ID NO: 2.

In one embodiment, the composition comprises a compound of Formula I:

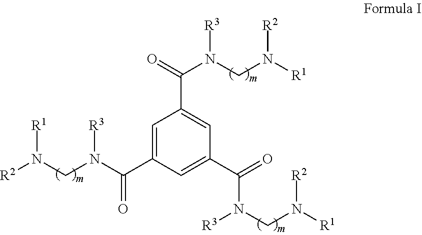

and salts thereof, wherein
each R¹ is independently hydrogen or substituted or unsubstituted alkyl;
each R² is independently substituted or unsubstituted alkyl;
each R³ is independently hydrogen or substituted or unsubstituted alkyl; and
each m is independently 1, 2, 3, 4, 5, 6, 7, or 8.

In one embodiment, the composition comprises a compound of Formula I:

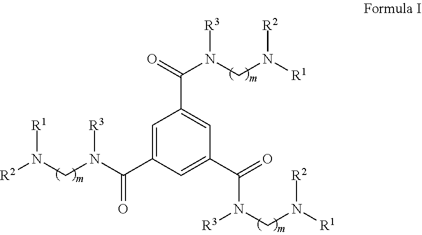

and salts thereof, wherein
each R¹ is independently unsubstituted alkyl;
each R² is independently unsubstituted alkyl;
each R³ is independently hydrogen or substituted or unsubstituted alkyl; and
each m is independently 3, 4, 5, 6, 7, or 8.

In one embodiment, the composition comprises a compound of Formula 1:

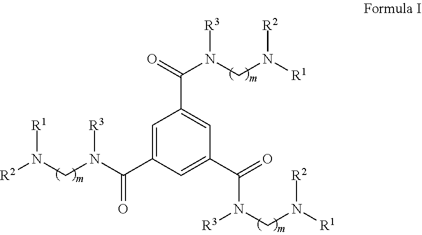

and salts thereof, wherein
each R¹ is independently unsubstituted alkyl;
each R² is independently unsubstituted alkyl;
each R³ is independently hydrogen or substituted or unsubstituted alkyl; and
each m is 3.

In some embodiments, at least one R¹ is substituted or unsubstituted C₁₋₂₄ alkyl. In some embodiments, at least one R¹ is substituted or unsubstituted C₁₋₁₈ alkyl. In some embodiments, at least one R¹ is substituted or unsubstituted C₁₋₁₂ alkyl. In some embodiments, at least one R¹ is substituted or unsubstituted C₆₋₁₈ alkyl. In some embodiments, at least one R¹ is substituted or unsubstituted C₆₋₁₂ alkyl. In some embodiments, at least one R¹ is substituted or unsubstituted C₈₋₁₂ alkyl. In some embodiments, at least one R¹ is substituted or unsubstituted C₁₀₋₁₂ alkyl. In some embodiments, at least one R¹ is unsubstituted C₁₀₋₁₂ alkyl. In some embodiments, at least one R¹ is substituted or unsubstituted Cu alkyl. In some embodiments, at least one R¹ is substituted or unsubstituted C₁₂ alkyl. In some embodiments, at least one R¹ is C₁₂H₂₅.

In some embodiments, at least one R² is substituted or unsubstituted C₁₋₂₄ alkyl. In some embodiments, at least one R² is substituted or unsubstituted C₁₋₁₈ alkyl. In some embodiments, at least one R² is substituted or unsubstituted C₁₋₁₂ alkyl. In some embodiments, at least one R² is substituted or unsubstituted C₆₋₁₈ alkyl. In some embodiments, at least one R² is substituted or unsubstituted C₆₋₁₂ alkyl. In some embodiments, at least one R² is substituted or unsubstituted C₈₋₁₂ alkyl. In some embodiments, at least one R² is substituted or unsubstituted C₁₀₋₁₂ alkyl. In some embodiments, at least one R² is unsubstituted C₁₀₋₁₂ alkyl. In some embodiments, at least one R² is substituted or unsubstituted Cu alkyl. In some embodiments, at least one R² is substituted or unsubstituted C₁₂ alkyl. In some embodiments, at least one R² is C₁₂H₂₅.

In some embodiments, at least two R¹ are substituted or unsubstituted C₁₋₂₄ alkyl. In some embodiments, at least two R¹ are substituted or unsubstituted C₁₋₁₈ alkyl. In some embodiments, at least two R¹ are substituted or unsubstituted C₁₋₁₂ alkyl. In some embodiments, at least two R¹ are substituted or unsubstituted C₆₋₁₈ alkyl. In some embodiments, at least two R¹ are substituted or unsubstituted C₆₋₁₂ alkyl. In some embodiments, at least two R¹ are substituted or unsubstituted C₈₋₁₂ alkyl. In some embodiments, at least two R¹ are substituted or unsubstituted C₁₀₋₁₂ alkyl. In some embodiments, at least two R¹ is unsubstituted C₁₀₋₁₂ alkyl. In some embodiments, at least two R¹ are substituted or unsubstituted C₁₁ alkyl. In some embodiments, at least two R¹ are substituted or unsubstituted C₁₂ alkyl. In some embodiments, at least two R¹ are C₁₂H₂₅.

In some embodiments, at least two R² are substituted or unsubstituted C₁₋₂₄ alkyl. In some embodiments, at least two R² are substituted or unsubstituted C₁₋₁₈ alkyl. In some embodiments, at least two R² are substituted or unsubstituted C₁₋₁₂ alkyl. In some embodiments, at least two R² are substituted or unsubstituted C₆₋₁₈ alkyl. In some embodiments, at least two R² are substituted or unsubstituted C₆₋₁₂ alkyl. In some embodiments, at least two R² are substituted or unsubstituted C₈₋₁₂ alkyl. In some embodiments, at least two R² are substituted or unsubstituted C₁₀₋₁₂ alkyl. In some embodiments, at least two R² is unsubstituted C₁₀₋₁₂ alkyl. In some embodiments, at least two R² are substituted or unsubstituted C₁₁ alkyl. In some embodiments, at least two R² are substituted or unsubstituted C₁₂ alkyl. In some embodiments, at least two R² are C₁₂H₂₅.

In some embodiments, all instances of R¹ are substituted or unsubstituted C₁₋₂₄ alkyl. In some embodiments, all instances of R¹ are substituted or unsubstituted C₁₋₁₈ alkyl. In some embodiments, all instances of R¹ are substituted or unsubstituted C₁₋₁₂ alkyl. In some embodiments, all instances of R¹ are substituted or unsubstituted C₆₋₁₈ alkyl. In some embodiments, all instances of R¹ are substituted or unsubstituted C₆₋₁₂ alkyl. In some embodiments, all instances of R¹ are substituted or unsubstituted C₈₋₁₂ alkyl. In some embodiments, all instances of R¹ are substituted or unsubstituted C₁₀₋₁₂ alkyl. In some embodiments, all instances of R¹ are unsubstituted C₁₀₋₁₂ alkyl. In some embodiments, all instances of R¹ are substituted or unsubstituted C₁₁ alkyl. In some embodiments, all instances of R¹ are substituted or unsubstituted C₁₂ alkyl. In some embodiments, all instances of R¹ are C₁₂H₂₅.

In some embodiments, all instances of R² are substituted or unsubstituted C₁₋₂₄ alkyl. In some embodiments, all instances of R² are substituted or unsubstituted C₁₋₁₈ alkyl. In some embodiments, all instances of R² are substituted or unsubstituted C₁₋₁₂ alkyl. In some embodiments, all instances of R² are substituted or unsubstituted C₆₋₁₈ alkyl. In some embodiments, all instances of R² are substituted or unsubstituted C₆₋₁₂ alkyl. In some embodiments, all instances of R² are substituted or unsubstituted C₈₋₁₂ alkyl. In some embodiments, all instances of R² are substituted or unsubstituted C₁₀₋₁₂ alkyl. In some embodiments, all instances of R² are unsubstituted C₁₀₋₁₂ alkyl. In some embodiments, all instances of R² are substituted or unsubstituted C₁₁ alkyl. In some embodiments, all instances of R² are substituted or unsubstituted C₁₂ alkyl. In some embodiments, all instances of R² are C₁₂H₂₅.

In some embodiments, at least one R¹ is substituted alkyl, wherein the substituted alkyl is substituted with a halogen. In some embodiments, at least one R¹ is substituted alkyl, wherein the substituted alkyl is substituted with fluorine. In some embodiments, at least one R¹ is substituted alkyl, wherein the substituted alkyl is substituted with halogenated alkyl.

In some embodiments, at least one R¹ is substituted alkyl, wherein the substituted alkyl is substituted with a halogen. In some embodiments, at least one R² is substituted alkyl, wherein the substituted alkyl is substituted with fluorine. In some embodiments, at least one R² is substituted alkyl, wherein the substituted alkyl is substituted with halogenated alkyl.

In some embodiments, at least two R¹ are substituted alkyl, wherein the substituted alkyl is substituted with a halogen. In some embodiments, at least two R¹ are substituted alkyl, wherein the substituted alkyl is substituted with fluorine. In some embodiments, at least two R¹ are substituted alkyl, wherein the substituted alkyl is substituted with halogenated alkyl.

In some embodiments, at least two R² are substituted alkyl, wherein the substituted alkyl is substituted with a halogen. In some embodiments, at least two R² are substituted alkyl, wherein the substituted alkyl is substituted with fluorine. In some embodiments, at least two R² are substituted alkyl, wherein the substituted alkyl is substituted with halogenated alkyl.

In some embodiments, all instances of R¹ are substituted alkyl, wherein the substituted alkyl is substituted with a halogen. In some embodiments, all instances of R¹ are substituted alkyl, wherein the substituted alkyl is substituted with fluorine. In some embodiments, all instances of R¹ are substituted alkyl, wherein the substituted alkyl is substituted with halogenated alkyl.

In some embodiments, all instances of R² are substituted alkyl, wherein the substituted alkyl is substituted with a halogen. In some embodiments, all instances of R² are substituted alkyl, wherein the substituted alkyl is substituted with fluorine. In some embodiments, all instances of R² are substituted alkyl, wherein the substituted alkyl is substituted with halogenated alkyl.

In some embodiments, at least one R³ is hydrogen. In some embodiments, at least one R³ is substituted or unsubstituted alkyl. In some embodiments, at least one R³ is substituted or unsubstituted C₁₋₁₈ alkyl. In some embodiments, at least one R³ is substituted or unsubstituted C₁₋₁₂ alkyl. In some embodiments, at least one R³ is substituted or unsubstituted C₁₋₆ alkyl. In some embodiments, at least one R³ is substituted or unsubstituted C₁₋₄ alkyl. In some embodiments, at least one R³ is substituted or unsubstituted C₂₋₄ alkyl. In some embodiments, at least one R³ is substituted or unsubstituted methyl.

In some embodiments, at least one R³ is substituted alkyl, wherein the substituted alkyl is substituted with a halogen. In some embodiments, at least one R³ is substituted alkyl, wherein the substituted alkyl is substituted with fluorine. In some embodiments, at least one R³ is substituted alkyl, wherein the substituted alkyl is substituted with halogenated alkyl.

In some embodiments, at least two R³ are hydrogen. In some embodiments, at least two R³ are substituted or unsubstituted alkyl. In some embodiments, at least two R³ are substituted or unsubstituted C₁₋₁₈ alkyl. In some embodiments, at least two R³ are substituted or unsubstituted C₁₋₁₂ alkyl. In some embodiments, at least two R³ are substituted or unsubstituted C₁₋₆ alkyl. In some embodiments, at least two R³ are substituted or unsubstituted C₁₋₄ alkyl. In some embodiments, at least two R³ are substituted or unsubstituted C₂₋₄ alkyl. In some embodiments, at least two R³ are substituted or unsubstituted methyl.

In some embodiments, at least two R³ are substituted alkyl, wherein the substituted alkyl is substituted with a halogen. In some embodiments, at least two R³ are substituted alkyl, wherein the substituted alkyl is substituted with fluorine. In some embodiments, at least two R³ are substituted alkyl, wherein the substituted alkyl is substituted with halogenated alkyl.

In some embodiments, all instances of R³ are hydrogen. In some embodiments, all instances of R³ are substituted or unsubstituted alkyl. In some embodiments, all instances of R³ are substituted or unsubstituted C₁₋₁₈ alkyl. In some embodiments, all instances of R³ are substituted or unsubstituted C₁₋₁₂ alkyl. In some embodiments, all instances of R³ are substituted or unsubstituted C₁₋₆ alkyl. In some embodiments, all instances of R³ are substituted or unsubstituted C₁₋₄ alkyl. In some embodiments, all instances of R³ are substituted or unsubstituted C₂₋₄ alkyl. In some embodiments, all instances of R³ are substituted or unsubstituted methyl.

In some embodiments, all instances of R³ are substituted alkyl, wherein the substituted alkyl is substituted with a halogen. In some embodiments, all instances of R³ are substituted alkyl, wherein the substituted alkyl is substituted with fluorine. In some embodiments, all instances of R³ are substituted alkyl, wherein the substituted alkyl is substituted with halogenated alkyl.

In some embodiments, at least one m is 1. In some embodiments, at least one m is 2. In some embodiments, at least one m is 3. In some embodiments, at least one m is 4. In some embodiments, at least one m is 5. In some embodiments, at least one m is 6. In some embodiments, at least one m is 7. In some embodiments, at least one m is 8.

In some embodiments, at least two m are 1. In some embodiments, at least two m are 2.

In some embodiments, at least two m are 3. In some embodiments, at least two m are 4. In some embodiments, at least two m are 5. In some embodiments, at least two m are 6. In some embodiments, at least two m are 7. In some embodiments, at least two m are 8.

In some embodiments, all instances of m are 1. In some embodiments, all instances of m are 2. In some embodiments, all instances of m are 3. In some embodiments, all instances of m are 4. In some embodiments, all instances of m are 5. In some embodiments, all instances of m are 6. In some embodiments, all instances of m are 7. In some embodiments, all instances of m are 8.

In some embodiments, the composition comprises a compound of Formula II:

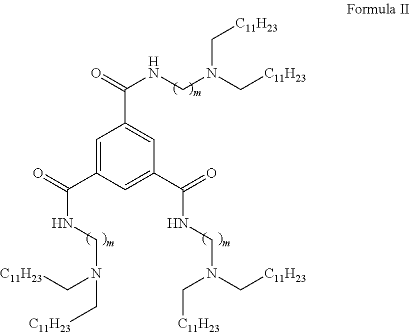

and salts thereof, wherein m=3.

In some embodiments, the lipid-like nanoparticle comprises a non-cationic lipid, a polyethylene glycol-lipid, and a sterol. In one embodiment, the non-cationic lipid can include, is a phosphatidylethanolamine lipid. In some embodiments, the phosphatidylethanolamine lipid is not limited to 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof.

In some embodiments, the nanoparticle can include a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs.

In some embodiments, the nanoparticle can be those found in, for example, WO2016/187531 or WO2019/099501.

In some embodiments, the nanoparticle can include a sterol. Sterols are well known to those skilled in the art and generally refers to those compounds having a perhydrocyclopentanophenanthrene ring system and having one or more OH substituents. In some embodiments, the sterol is selected from cholesterol, campesterol, ergosterol, sitosterol and the like.

### Methods

In one aspect, disclosed herein is a method of treating a cancer in a subject comprising administering a composition comprising a lipid-like nanoparticle encapsulating a CCL5 mRNA.

In one aspect, disclosed herein a composition comprising a lipid-like nanoparticle encapsulating a CCL5 mRNA. In some embodiments, the CCL5 mRNA is encoded by SEQ ID NO: 1 or SEQ ID NO: 4. In some embodiments, the CCL5 mRNA encodes a protein comprising SEQ ID NO: 2 or SEQ ID NO: 5. In some embodiments, the CCL5 mRNA comprises SEQ ID NO: 3 or SEQ ID NO: 6.

In some embodiments, the CCL5 mRNA is encoded by a sequence at least 60% (for example, at least 60%, at least 65%, at least 70%, at least 75%, at least 80%, at least 85%, at least 90%, at least 95%, or more) identical to SEQ ID NO: 1. In some embodiments, the CCL5 mRNA is encoded by a sequence at least 60% (for example, at least 60%, at least 65%, at least 70%, at least 75%, at least 80%, at least 85%, at least 90%, at least 95%, or more) identical to SEQ ID NO: 4.

In some embodiments, the CCL5 mRNA encodes a protein sequence at least 60% (for example, at least 60%, at least 65%, at least 70%, at least 75%, at least 80%, at least 85%, at least 90%, at least 95%, or more) identical to SEQ ID NO: 2. In some embodiments, the CCL5 mRNA encodes a protein sequence at least 60% (for example, at least 60%, at least 65%, at least 70%, at least 75%, at least 80%, at least 85%, at least 90%, at least 95%, or more) identical to SEQ ID NO: 5.

In some embodiments, the CCL5 mRNA comprises a sequence at least 60% (for example, at least 60%, at least 65%, at least 70%, at least 75%, at least 80%, at least 85%, at least 90%, at least 95%, or more) identical to SEQ ID NO: 3. In some embodiments, the CCL5 mRNA comprises a sequence at least 60% (for example, at least 60%, at least 65%, at least 70%, at least 75%, at least 80%, at least 85%, at least 90%, at least 95%, or more) identical to SEQ ID NO: 6.

In some embodiments, the subject is further administered an anticancer agent. In some embodiments, the anticancer agent is an immunotherapeutic agent. In some embodiments, the anticancer agent is selected from a PD-L1 antibody, a PD-1 antibody, or a CTLA-4 antibody. In some embodiments, the anticancer agent is a PD-L1 antibody. In some embodiments, the anticancer agent is a PD-1 antibody. In some embodiments, the anticancer agent is a CTLA-4 antibody.

In some embodiments, the lipid-like nanoparticle comprises a compound of Formula I:

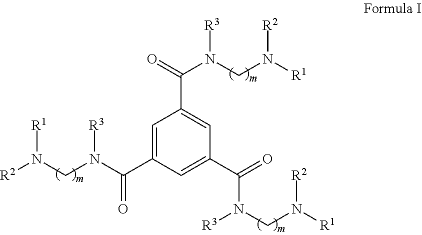

and salt thereof, wherein R¹, R², R³, and m are described herein.

In some embodiments, the method comprises the lipid-like nanoparticle comprises a compound of Formula II:

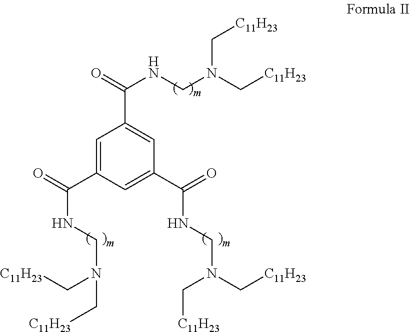

and salts thereof.

In some embodiments, the method comprises the lipid-like nanoparticle further comprising a non-cationic lipid, a polyethylene glycol-lipid, and a sterol. In one embodiment, the non-cationic lipid can include a phosphatidylethanolamine lipid. In some embodiments, the phosphatidylethanolamine lipid is not limited to 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In some embodiments, the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs. In some embodiments, the sterol is selected from cholesterol, campesterol, ergosterol, or sitosterol.

In some embodiments, the cancer is a skin cancer. In some embodiments, the skin cancer is a basal cell carcinoma. In some embodiments, the skin cancer is a squamous cell carcinoma. In some embodiments, the skin cancer is a Merkel cell cancer. In some embodiments, the skin cancer is a melanoma. In some embodiments, the cancer is a breast cancer. In some embodiments, the cancer is a cervical cancer. In some embodiments, the cancer is a liver cancer. In some embodiments, the cancer is a lung cancer. In some embodiments, the cancer is an uterine cancer. In some embodiments, the cancer is a prostate cancer. In some embodiments, the cancer is an ovarian cancer. In some embodiments, the cancer is a renal cancer. In some embodiments, the cancer is a thyroid cancer.

In addition to enhancing NK and T cell functions, CCL5 can recruit Tregs into tumors. Thus, intratumor injection of NP-CCL5 mRNA can cause accumulation of Tregs in tumors, which diminishes T cell anti-tumor activity. However, this effect can be counteracted by anti-CTLA4 antibody therapy, which can selectively deplete Tregs from tumors. Thus, in some embodiments, NP-CCL5 mRNA+anti-CTLA4 combination therapy can also be used for cancer therapy.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. IL-27 Induces CCL5 Production by T Lymphocytes which Contributes to Anti-Tumor Activity

IL-27 is a member of the IL-12 cytokine family that consists of an IL-12 p40-related protein subunit, EBV-induced gene 3 (EBI3), and a p35-related subunit, p28. IL-27 signals through a heterodimeric receptor (IL-27R) composed of the WSX-1 (IL-27Rα) and the gp130 subunits in a variety of cell types, including T lymphocytes. IL-27R signaling enhances the recruitment of several JAK family kinases and activates STAT family transcription factors 1 and 3. IL-27 enhances Th1/Tel responses by activating the Stat1-T-bet axis and promotes T cell expression of T-bet, Eomes, IL-12Rβ2, granzyme B and Perforin. It also inhibits Th2 and Th17 responses by blocking the expression of transcription factors GATA-3 (Th2) and RoRγτ (Th17) and is an inducer of IL-10 production by T cells. Moreover, IL-27 can also induce PD-L1 I expression in T cells which restrain T cell effector functions by interacting with PD-1 on T cells. These functional properties of IL-27 show that it enhances anti-tumor immunity and inhibits Th17/Th2 mediated autoimmunity.

The therapeutic effects of IL-27 delivered by adeno-associated virus (AAV-IL-27) was evaluated in mouse tumor models. These studies revealed that first, AAV-IL-27 significantly inhibited the growth of a broad spectrum of tumor types in mice with low or no toxicity. Second, AAV-IL-27 treatment resulted in dramatic reduction of Tregs without causing autoimmunity. Third, AAV-IL-27 therapy shows strong synergy with PD-1 antibody or cancer vaccines in inhibiting tumor growth. In searching for IL-27 induced genes in T cells, C—C motif chemokine ligand 5 (CCL5), also known as RANTES, was found to be most significantly upregulated. CCL5 is a member of the CC chemokine family and is detected in multiple cell types including immune cells such as NK cells, T cells, dendritic cells (DCs) and macrophages. In tumor microenvironment (TME), cancer cells also serve as a source of CCL5. CCL5 works primarily through interaction with CCR5 receptor, but it can also engage other receptors such as CCR1 and CCR3. Functionally, CCL5 is a proinflammatory chemokine recruiting various leukocytes including T, NK, and DCs to the site of inflammation. However, the role of CCL5 in tumor immunity remains controversial. While some studies show that CCL5 production leads to a more immune suppressive TME, other evidence demonstrated that CCL5 in TME is in favor of tumor immunity.

Here, it was demonstrated that IL-27 directly induces CCL5 production by T lymphocytes, particularly CD8⁺ T cells in vitro and in vivo. IL-27-induced CCL5 production is IL-27R-dependent and requires both Stat3 and Stat1 signaling. In CD4⁺ T cells, IL-27 induced CCL5 production is primarily dependent on Stat1 activation, while in CD8⁺ T cells, Stat1-deficiency does not abrogate CCL5 induction. Chromatin immunoprecipitation (ChIP) assay reveals that Stat3 to be the dominant mediator of IL-27-induced induction of CCL5 in CD8⁺ T cells, since both putative Stat3 binding sites exhibited significant binding of Stat3, while only one out of four Stat1 binding sites displayed moderate binding to Stat1. In IL-27 treated tumor-bearing mice, IL-27 induced dramatic production of CCL5 in tumor-infiltrating T cells. IL-27-induced CCL5 appears to contribute to IL-27-mediated anti-tumor effect, as significantly less tumor inhibition was observed in anti-CCL5, and IL-27 treated mice.

### Materials and Methods

Mice: CD45.1 congenic C57BL6, C57BL/6, IL27R^{-/-}C57BL6 and BALB/c mice were originally purchased from The Jackson Laboratory. Stat1^{-/-} BALB/c mice and P1CTL TCR transgenic mice were described previously. These mouse strains were maintained in the animal facilities of the Ohio State University (OSU). All animal protocols were approved by the OSU Animal Care and Use Committee (Approved IACUC protocol 2008A0093-R4) and mice were treated in accordance with institutional guidelines for animal care. The OSU Laboratory Animal Shared Resource is an Association for Assessment and Accreditation of Laboratory Animal Care International accredited program that follows Public Health Service policy and guidelines.

Preparation of CD4⁺ and CD8⁺ T cells and in vitro activation: To prepare CD4⁺ and CD8⁺ T cells, mouse spleen and lymph nodes were processed into single-cell suspensions. CD4⁺ and CD8⁺ cells were purified by positive selection using anti-CD4 or anti-CD8-PE and anti-PE-magnetic microbeads on a MACS column (Miltenyi Biotech, Germany) Positive selection was also used to purify human CD8⁺ T cells from peripheral blood mononuclear cells. Purified CD4⁺ or CD8⁺ T cells were incubated with anti-CD3/CD28 beads (Dynabeads Mouse T-Activator CD3/28, Thermo Fisher) in click's EHAA medium (Invitrogen) containing 100 μg/mL penicillin and 100 μg/mL streptomycin, 1 mM 2-ME, 5% fetal bovine serum, in the absence or presence of 50 ng/ml recombinant IL-27 (Biolegend) for up to 5 days. To activate P1CTL T cells, 0.3×10⁶/mL spleen cells from P1CTL TCR transgenic mice were stimulated with 0.1 μg/mL P1A35-43 peptide in EHAA medium in the absence or presence of 50 ng/ml recombinant mouse IL-27 for up to 5 days.

Production of rAAV and treatment of mice: rAAV-mIL-27 and rAAV-ctrl viruses were produced as described previously. AAV viruses were diluted in PBS and injected intramuscularly (i.m.) into two sites of hind legs in a total volume of 100 l, containing 2×10¹¹ DNase-resistant particles (DRP) of AAV.

Flow cytometry: FITC-, PE-, PE-CY7, APC-, APC-CY7 or Percp-Cyanine5.5 labeled antibodies to mouse CCL5 (2E9/CCL5), CD45.1 (A20), CD4 (GK1.5), CD8α (53-6.7), CD11b (M1/70), CD11c (N418), NK1.1 (PK136) and isotype-matched control antibodies were purchased from Biolegend or BD Biosciences. APC-labeled antibody to human CD8 (RPA-T8) and CCL5 (VL1) were purchased from BD Biosciences. For identification of cellular phenotypes, disassociated cells from spleens or tumors were re-suspended in PBS containing 1% bovine serum albumin and incubated with the antibodies on ice for 30 minutes. Cells were fixed in 1% paraformaldehyde in PBS after washing. For intracellular CCL5 staining, viable cells were fixed and permeabilized with transcription staining buffer set (eBioscience) and stained with respective antibody. Stained cells were analyzed on a FACSCalibur or FACS Celesta flow cytometer, and data were analyzed using the Flowjo software.

Determination of IL-27 induced CCL5 mRNA expression in P1CTL cells: Spleen cells from P1CTL mice were cultured in Click's EHAA medium containing 0.1 μg/mL of P1A35-43 in the presence or absence of mIL-27 (50 ng/ml). The total RNA was extracted at day 1, 2, 3, 4, and 5 after T cell culture using Qiagen RNeasy Mini kit following the manufacturer's protocol. The expression levels of CCL5 mRNA were determined by qRT-PCR using primers specified in Table 1.

Chromatin Immunoprecipitation (ChIP): P1CTL cells were activated with PIA peptide with or without IL-27 for four days as described above. Then the resulting CTLs were fixed and sheared by sonication. ChIP was performed using ChIP-IT® Express Kits (Active Motif, 53008) according to the manufacturer's protocol. The following antibodies, including anti-STAT1 (Cell Signaling #9172), anti-STAT3 (Santa Cruz, sc-428), anti-RNA polymerase II (Abcam, ab5153), and a control antibody (Abcam, ab171870) were used in precipitation. 25 μg sheared chromatin was used in each precipitation reaction. Selective immunoprecipitation of CCL5 promoter fragments was determined by qPCR using the high-capacity cDNA reverse transcription kit (Applied Biosystems, 4368813) and the primers are specified in Table 1. Input % was calculated by ChIP/Input×100%.

Tumor establishment in mice and treatments with AAV-IL-27 and anti-CCL5 antibody: C57BL6 mice were injected with B16.F10 melanoma cells, which were originally obtained from ATCC, and maintained in RPMI1640 (Gibco) medium supplemented with 100 μg/ml penicillin, 100 μg/ml streptomycin, and 10% FBS (Gibco). To establish tumors in mice, 1×10⁵ B16.F10 cells were injected into each C57BL6 mouse s.c. in 100 μl of PBS. Four days after tumor inoculation, the mice received i.m. injection of AAV-IL-27 or AAV-ctrl virus at a dose of 2×10¹¹ DRP per mouse. On day 13, 17, 22 and 26 mice were also treated with 100 mg/mouse of anti-CCL5 (Clone #53405, R&D system) or a control IgG2a antibody (BioXcell) i.p. The length and width of tumors were measured using a digital caliper every 2 days. The tumor volume was calculated according to the formula volume (V)=ab²/2, where a represents length and b represents width of tumors.

CCL5 mRNA synthesis and encapsulation into nanoparticles: CCL5 expression plasmid was purchased from InvivoGen (San Diego, CA, USA) and was used as a template for in vitro transcription. mRNAs were synthesized with full substitution of UTP by pseudouridine-5′-triphosphate (TriLink, USA) using AmpliScribe T7-Flash Transcription Kit (Lucigen, USA). The resulting mRNA was purified by RNA Clean & Concentrator (Zymo, USA) and capped using Vaccinia Capping System (NEB, USA) and Cap 2′-O-Methyltransferase (NEB, USA). Purified CCL5 mRNA was quantified using a NanoDrop 2000 Spectrophotometer (ThermoFisher, USA), and was mixed with lipid nanoparticles (NP) to prepare NP-CCL5 mRNA using a method described before.

Treatment of mice via intratumoral injection of NP-CCL5 mRNA: C57BL6 mice were inoculated with B16.F10 melanoma cells (1×10⁵ cells/mouse) s.c. When tumors were fully established on day 10, mice started to receive intratumor injection of either NP-CCL5 mRNA (2 mg/mouse in 50 ml PBS) or vehicle only every other day for 6 times. Tumor size was measured every two days and tumor volumes were calculated.

Statistical Analysis: One way ANOVA and student's t test were used for comparison among multiple groups or between two groups. The GraphPad Prism software 8.0 was used for ANOVA and t-tests.

### Results

### 1. IL-27 Induces CCL5 Production in Mouse and Human T Cells.

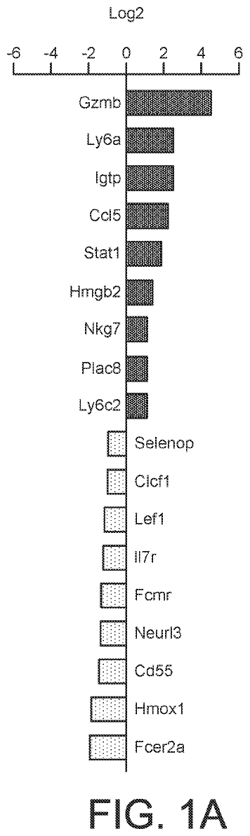

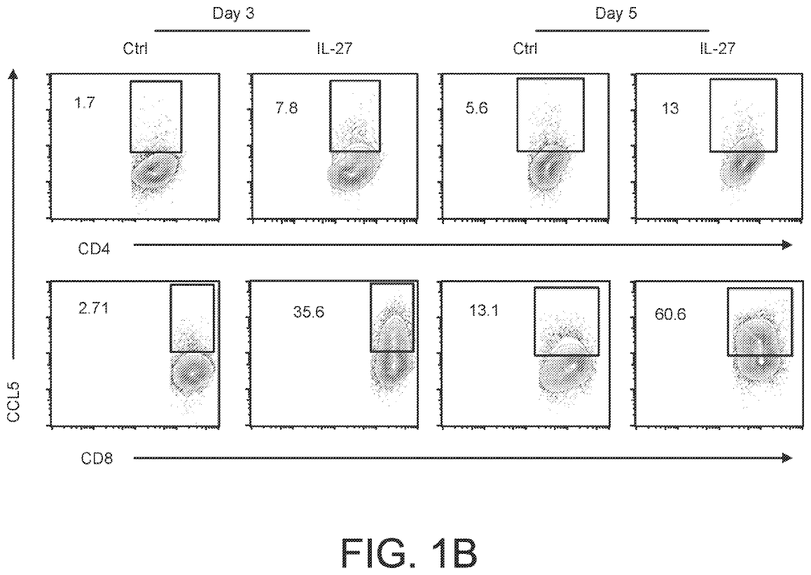

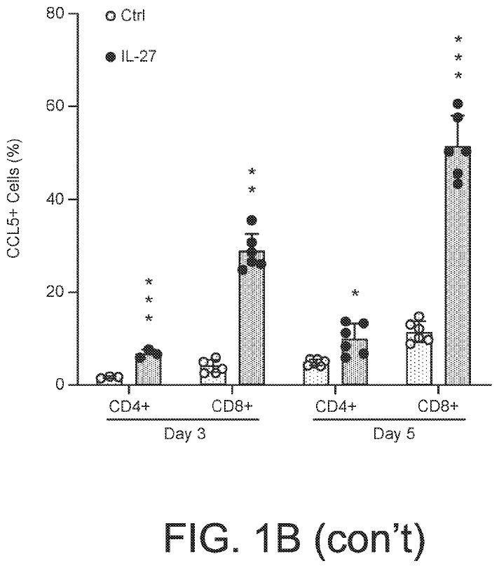

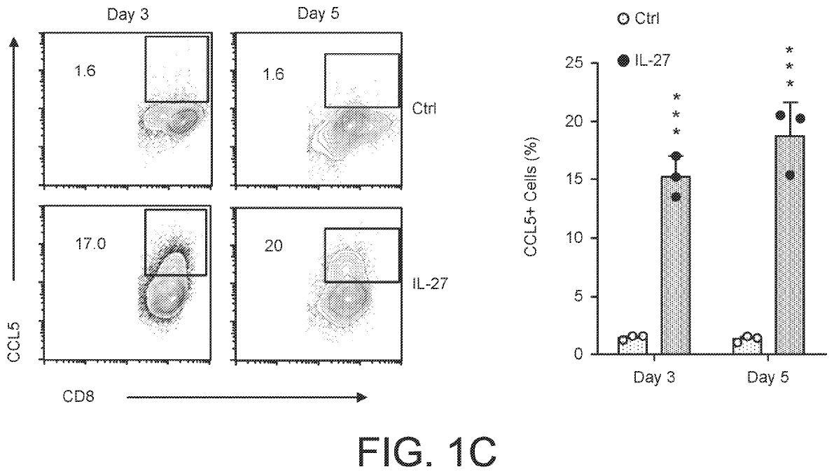

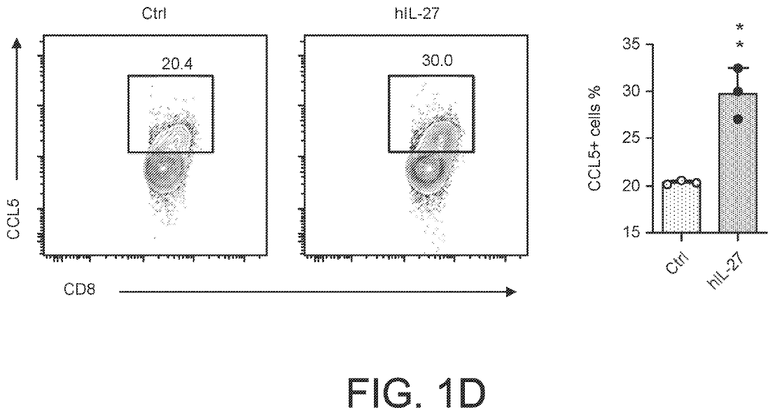

The use of AAV-IL-27 as a therapeutic was evaluated in mouse tumor models. These studies revealed that AAV-IL-27 significantly inhibited the growth of a broad spectrum of tumor types in mice with low or no toxicity. To understand pathways induced by IL-27, RNAseq analysis was performed on CD8⁺ T cells from mice treated with AAV-IL-27 (GSE195736; www.ncbi.nlm.nih.gov/geo/info/linking.html). One of the most significantly upregulated genes was found to be CCL5 (FIG. 1A). To validate this observation, naïve CD4⁺ and CD8⁺ T cells were purified from mouse spleen and lymph nodes and activated T cells with anti-CD3/CD28 beads in the presence or absence of IL-27. As shown in FIG. 1B, IL-27 upregulated CCL5 production in both CD4⁺ and CD8⁺ T cells three or five days after culture. However, IL-27 most significantly upregulated CCL5 in CD8⁺ T cells five days after activation. P1CTL TCR transgenic T cells were activated with PIA peptide with or without IL-27, then analyzed CCL5 production using flow cytometry. As shown in FIG. 1C, IL-27 stimulation significantly upregulated CCL5 production in P1CTL T cells. Similarly, purified human CD8⁺ T cells were activated with anti-CD3/CD28 beads in the presence or absence of IL-27. IL-27 also upregulated CCL5 production in human CD8⁺ T cells (FIG. 1D).

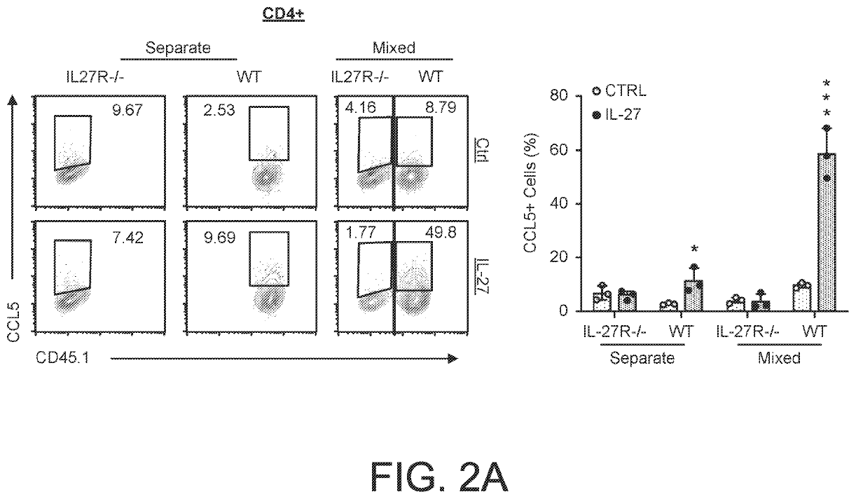

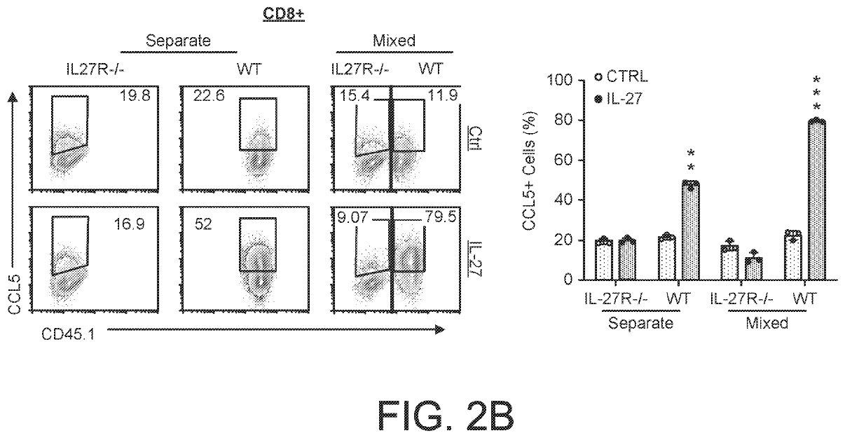

To determine if IL-27 directly induces CCL5 production in T cells, naïve CD4⁺ and CD8⁺ T cells were purified from IL27Rα^{-/-} and WT mice, and stimulated T cells with anti-CD3/CD28 with or without IL-27. As shown in FIG. 2, IL-27 only induced CCL5 in T cells from WT but not IL-27Rα^{-/-} mice. Additionally, when T cells from IL27Rα^{-/-} mice were mixed with T cells from WT mice at 1:1 ratio followed by activation, IL-27 only induced CCL5 in CD4⁺ (FIG. 2A) and CD8⁺ (FIG. 2B) T cells from WT, but not CD27Rα^{-/-} T cells. Thus, IL-27 induces CCL5 production in T cells through direct stimulation of IL27R on T cells, but not through bystander factors induced by IL-27 and T cell activation.

### 2. Differential Involvement of Stat1 and Stat3 in IL-27-Induced CCL5 Production in T Cells

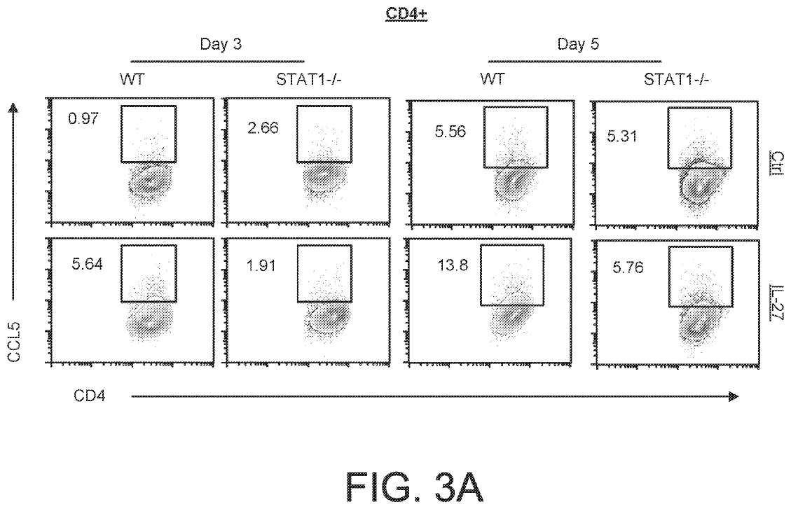

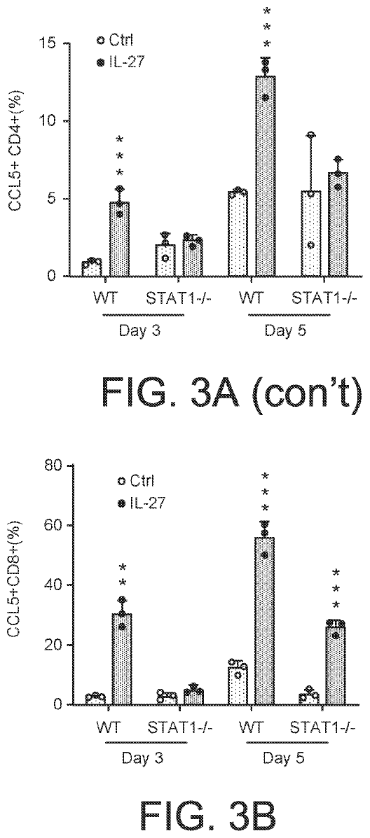

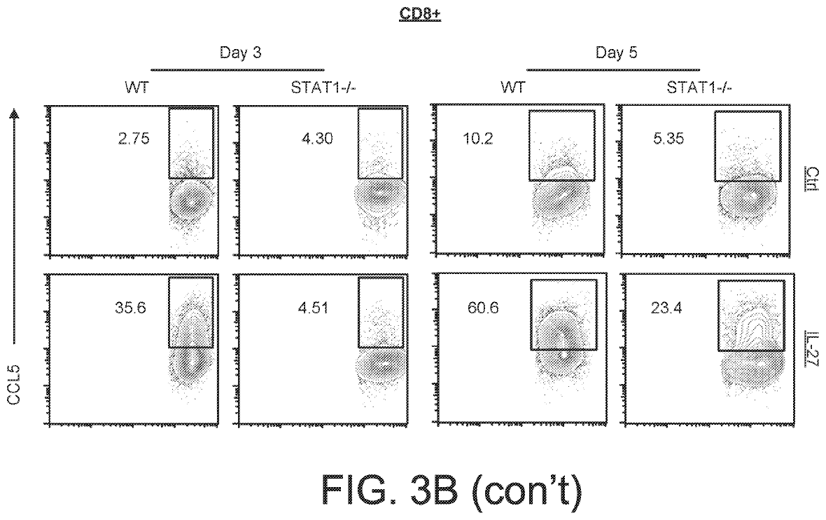

IL-27 signals through IL-27R and activates Stat1 and Stat3 (3, 4). Therefore, IL-27 induction of CCL5 through Stat1 or Stat3 was tested. As shown in FIG. 3A, while IL-27 induced significant CCL5 on day 3 and day 5 in CD4⁺ T cells, Stat1-deficiency abrogated CCL5 induction in CD4⁺ T cells. For CD8⁺ T cells, Stat1-deficiency also significantly diminished CCL5 induction on day 3 and day 5. However, significant CCL5 induction by IL-27 was still evident on day 5 after T cell activation (FIG. 3B). Thus, it appears that IL-27-induced CCL4 production in CD4⁺ and CD8⁺ T cells is dependent on Stat1, while in CD8⁺ T cells, Stat3 is also likely involved.

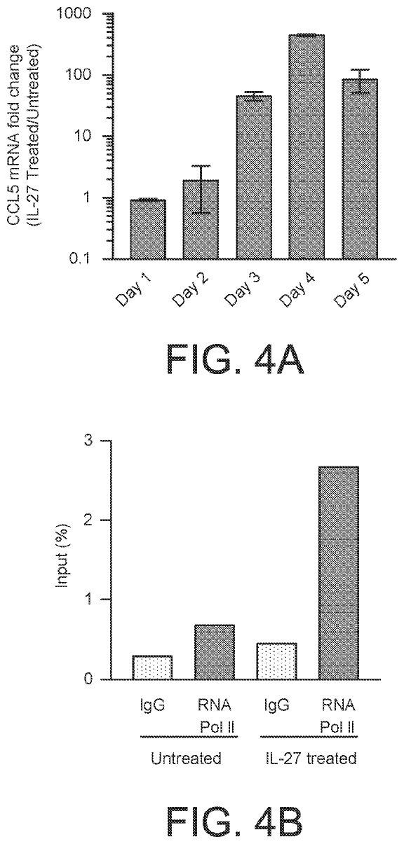

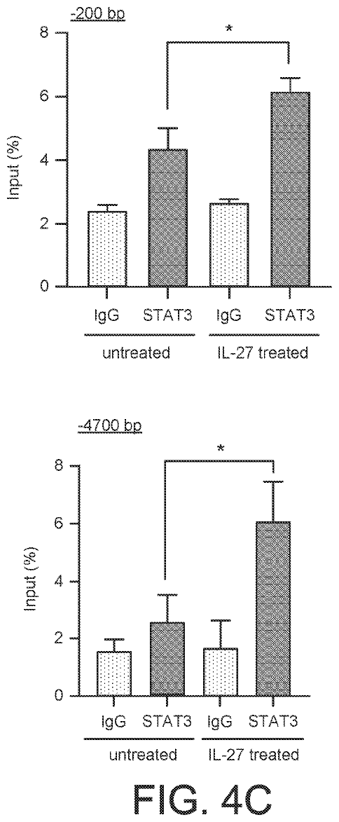

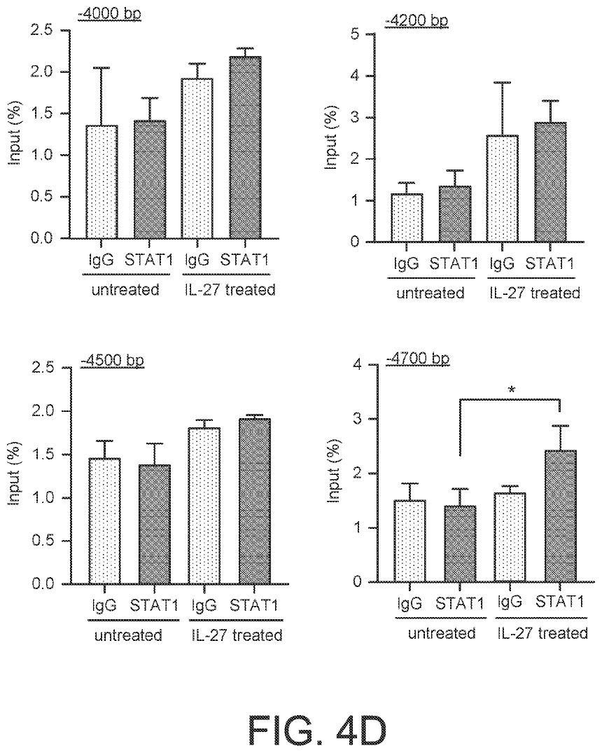

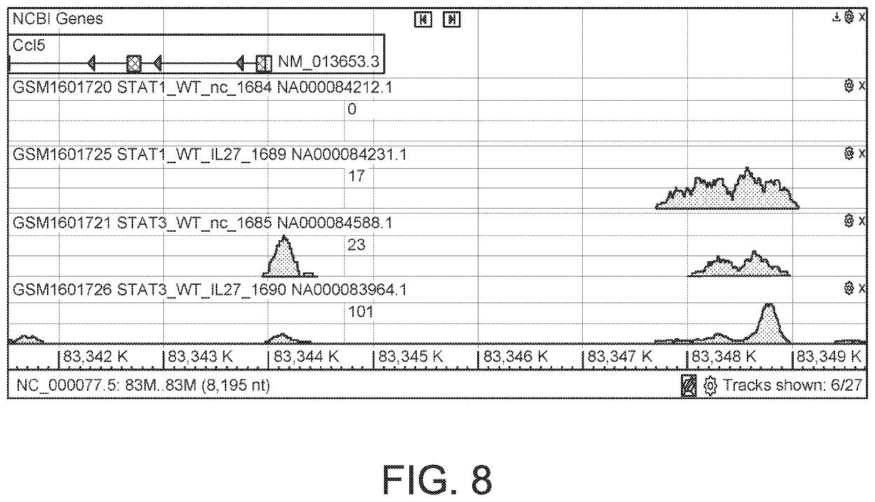

To determine the peak of CCL5 mRNA expression in the presence of IL-27, primary mouse P1CTL cells were activated with PIA peptide in the presence or absence of IL-27 for 1 to 5 days. The relative expression of CCL5 mRNA was determined by RT-qPCR. As shown in FIG. 4A, dramatic induction of IL-27 mRNA was detected through day 3-5, with day 4 as the peak Recently, the global roles of STAT1 and STAT3 in IL-27-induced transcriptomic changes were revealed by ChIP-seq using anti-STAT1 and anti-STAT3 antibodies. Therefore, ChIP-seq data deposited to Gene Expression Omnibus (Accession Number GSE65621) was examined. Close examination of the region upstream of CCL5 transcription start site (TSS) revealed several putative STAT1 and STAT3 binding sites (FIG. 8). Based on the ChIP-seq data, STAT1 possibly binds to the region spanning from −4000 bp to −4800 bp relative to CCL5 TSS, while STAT3 may bind to a −200 bp site and a −4700 bp site. To confirm STAT1 and/or STAT3 binding, primary mouse P1CTL cells were activated with PIA peptide for 4 days with and without IL-27. Then ChIP assay was performed on P1CTL cells using qPCR primers designed to probe the putative binding sites (Table 1). In controls, IL-27 stimulation enhanced RNA polymerase II's binding to the promoter of CCL5 (FIG. 4B). Increased STAT3 binding were detected at both the −200 bp and −4700 bp sites upstream of CCL5 TSS (FIG. 4C), while significantly increased STAT1 binding was detected in one of the four binding sites (−4700 bp site) (FIG. 4D). These results show that while both STAT1 and STAT3 are involved in IL-27 induced CCL5 induction, STAT3 activation is more evident in the CCL5 promotor of CD8⁺ T cells.

### 3. IL-27 Treatment Upregulates CCL5 in T Cells In Vivo

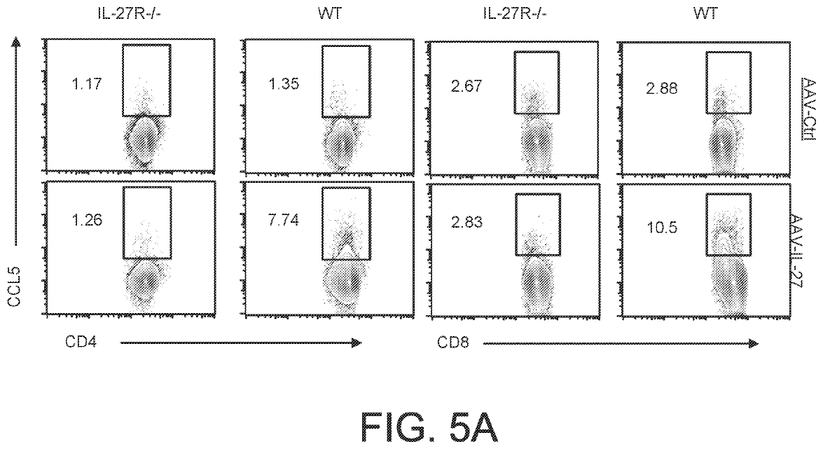

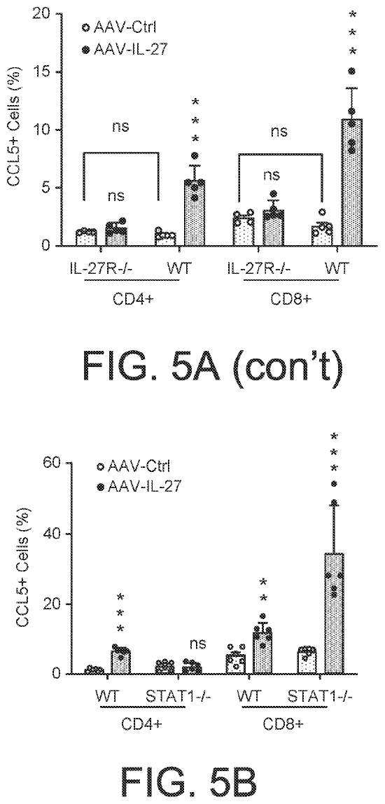

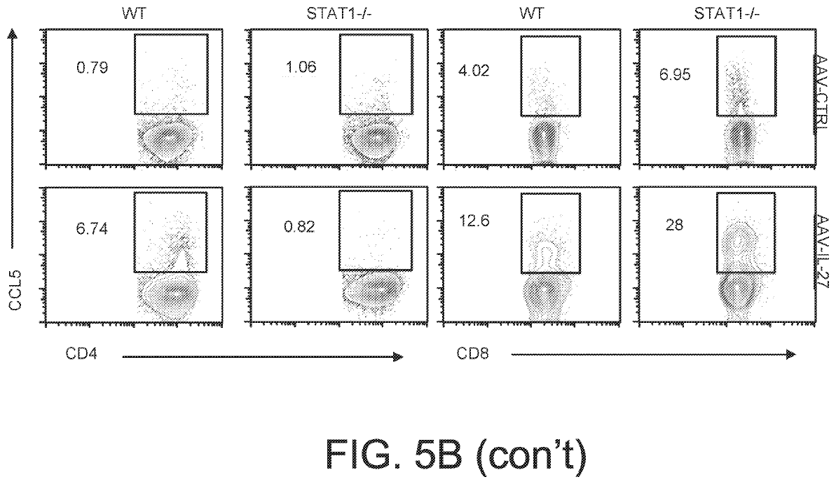

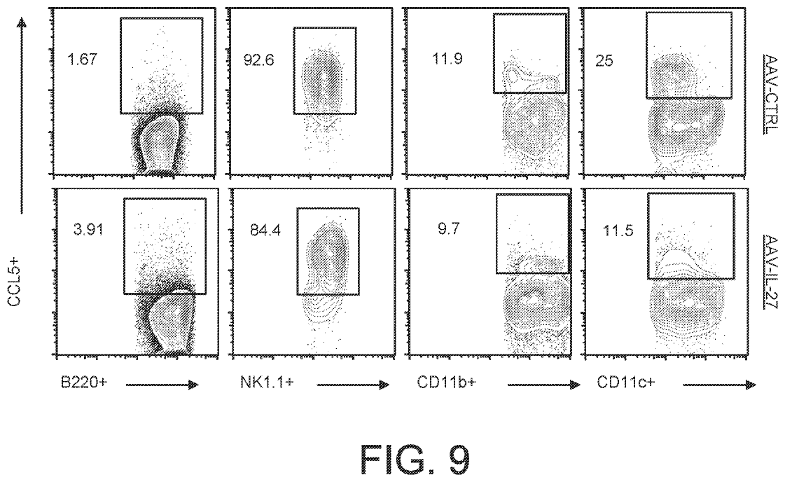

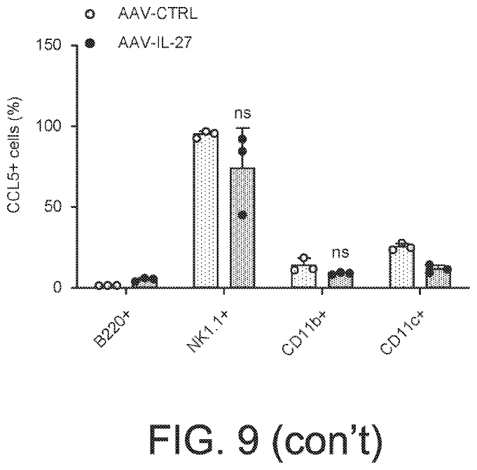

To determine if IL-27 also induces CCL5 induction in T cells in vivo, IL27Rα^{-/-} and Stat1^{-/-} mice and their relative control mice were treated with AAV-IL-27 or AAV-ctrl virus at a dose of 2×10¹¹ DRP/mouse i.m. Three weeks after viral treatment, mice were sacrificed and expression of CCL5 in spleen T cells were analyzed by flow cytometry. As shown in FIG. 5A, treatment with AAV-IL-27 significantly induced CCL5 in both CD4⁺ and CD8⁺ spleen T cells from WT, but not IL-27Rα^{-/-} mice, while treatment with AAV-ctrl failed to induce CCL5 in both WT and IL27Rα^{-/-} mice. Additionally, STAT1-deficiency abrogated IL-27-induced CCL5 production in CD4⁺ T cells, but not in CD8⁺ T cells (FIG. 5B). In AAV-IL-27 treated mice, AAV-IL-27 treatment induction of CCL5 in other splenic leukocytes was also evaluated. AAV-IL-27 treatment failed to induce CCL5 in cells including B cells, NK cells, CD11b⁺ monocytes and CD11c⁺ dendritic cells (FIG. 9), demonstrating that IL-27 alone is insufficient to induce CCL5 in these cell types.

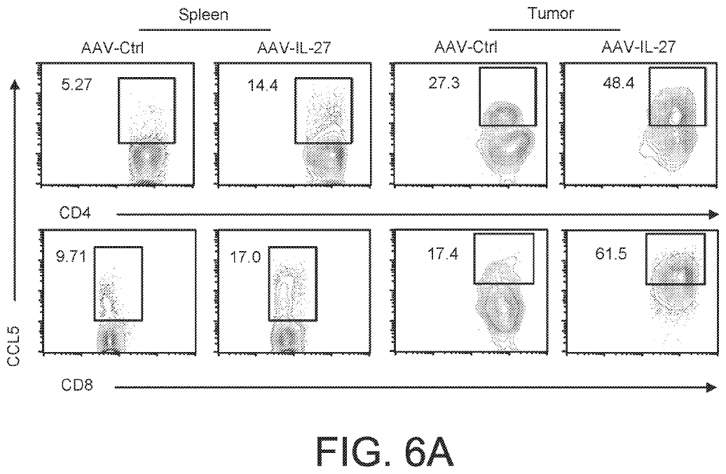

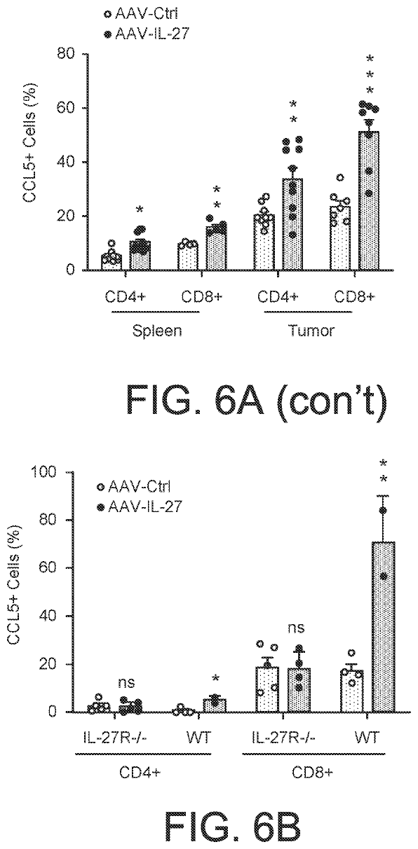

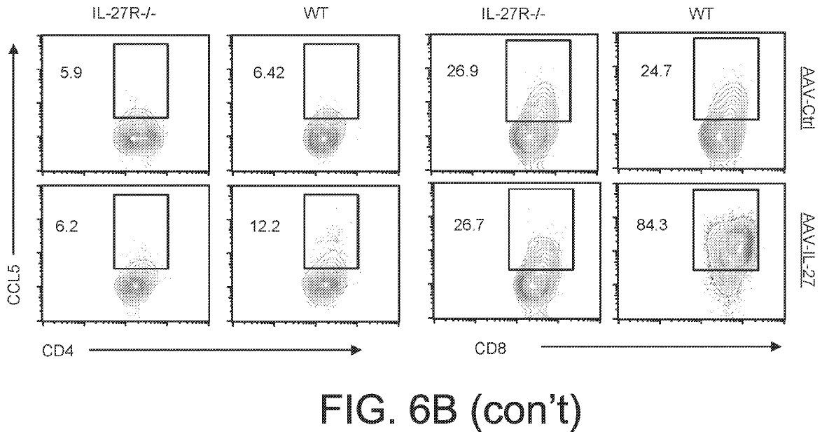

To determine CCL5 induction by IL-27 in tumor-bearing mice, WT and IL27Rα^{-/-} mice were injected with 1×10⁵ cells/mouse of B16F10 cells s.c. Four days later, mice were treated with AAV-IL-27 or AAV-ctrl virus at a dose of 2×10¹¹ DRP/mouse i.m. Three weeks after viral treatment, mice were sacrificed and expression of CCL5 in spleen T cells and tumors were analyzed by flow cytometry. As shown in FIG. 6A, AAV-IL-27 treatment induced CCL5 in both spleen T cells and tumor-infiltrating T cells, while much higher CCL5 induction was observed in tumor-infiltrating T cells. In both spleens and tumors, AAV-IL-27 induction of CCL5 was strictly IL-27R-dependent (FIG. 6B).

### 4. IL-27-Induced CCL5 Inhibits Tumor Growth

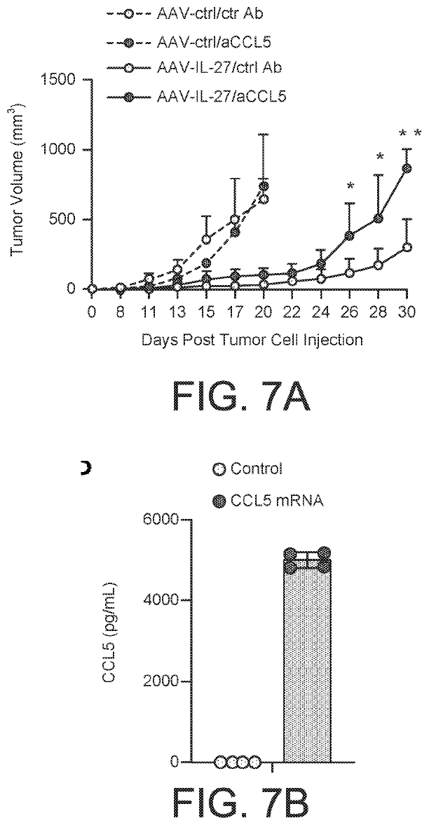

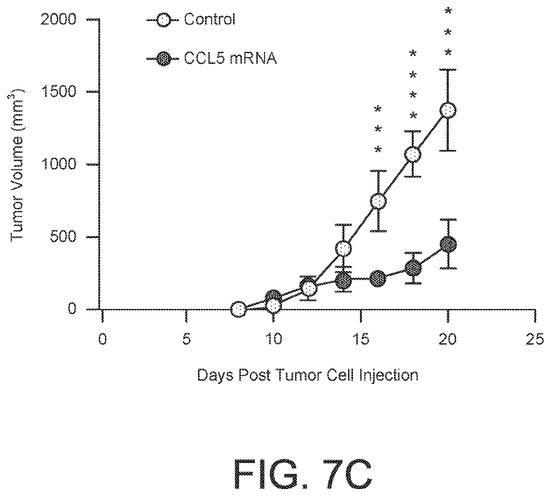

Previous reports concerning the role of CCL5 in tumors were controversial. While some studies suggest that CCL5 production may lead to a more immune suppressive TME, other evidence suggests that CCL5 in TME is in favor of tumor immunity. To determine the role of IL-27 induced CCL5 in tumor immunity, the following two sets of experiments were performed. First, C57BL6 mice were initially inoculated with B16.F10 tumor cells s.c. followed by treatment with AAV-IL-27 or AAV-ctrl virus (2×10¹¹ DRP/mouse i.m.) on day 4. Then, mice were also treated with anti-CCL5 (100 mg/mouse) or a control IgG2a antibody (100 mg/mouse) i.p. on days 13, 17, 22 and 26. As shown in FIG. 7A, while mice treated with AAV-ctrl/anti-CCL5 and AAV-ctrl/ctrl Ab exhibited similar tumor growth kinetics, anti-CCL5 treatment significantly reduced the efficacy of AAV-IL-27-mediated tumor inhibition. This conclusion was the result of significantly larger tumor volumes measured on days 26, 28 and 30. Second, lipid nanoparticles (NP) encapsulated with CCL5 mRNA (NP-CCL5 mRNA) were generated. In vitro cell culture experiments revealed that NP mediated delivery of CCL5 mRNA efficiently translated into CCL5 protein in B16.F10 cells (FIG. 7B) As shown in FIG. 7C, intratumoral injection of NP-CCL5 mRNA significantly inhibited tumor growth, as measured by significantly reduced mean tumor volume. Thus, neutralizing CCL5 compromised AAV-IL-27-mediated antitumor activity, while intratumor delivery of CCL5 mRNA directly inhibited tumor growth.

### Discussion

There is an increasing body of literature for IL-27 regulating lymphocyte chemotaxis by modulating expression of chemokine/chemokine receptors. IL-27 was found to induce the release of CCL2, CXCL9 and CXCL10 in human primary fibroblast-like synoviocytes. Here, it was found that IL-27 directly upregulates CXCR3 in T cells, which is the receptor for CXCL9 and CXCL10. Another study reported that IL-27 signaling suppresses splenic CD4⁺ T cell CCR5-dependent chemotactic responses during infection through restricting CCR5 expression. Interestingly, IL-27-treatment during influenza reduced secretion level of multiple chemokines including CXCL1, CCL4 and CCL5 by CD11b⁺ macrophages and CD11c⁺ myeloid cells. In contrast to this observation, it was reported that pretreatment with IL-27 followed by stimulation with TL8, R848, or CL075 significantly enhanced production of IL-8 and CCL5 in macrophages. In this work, it is demonstrated that IL-27 directly induces CCL5 production in both mouse and human T cells, in particular CD8⁺ T cells in vitro and in vivo. Thus, this current study reveals a novel function of IL-27 in T cells.

In this work, IL-27 was observed to induce CCL5 production only in WT but not IL-27Rα^{-/-} T cells even when IL-27Rα^{-/-} and WT T cells were mixed, showing that no bystander effect is involved, and IL-27 directly induces CCL5 production by T cells. In CD4⁺ T cells, it was observed that IL-27 induced CCL5 production is primarily dependent on Stat1 activation in vitro (FIG. 3) and in vivo (FIG. 5). However, in CD8⁺ T cells, Stat1-deficiency reduced, but did not abrogate CCL5 induction in vitro (FIG. 3). In vivo, IL-27-induced CCL5 production by CD8⁺ T cells was not significantly affected by Stat1-deficiency (FIG. 5). ChIP assay also revealed both Stat1 and Stat3 involvement. STAT3 appears to be the dominant mediator of IL-27-induced activation of CCL5 in CD8⁺ T cells (FIG. 4). Thus, this study reveals a mechanism of IL-27 induction of CCL5 in T cells: in CD4⁺ T cells, CCL5 induction is Stat1-dependent, while in CD8⁺ T cells, induction of CCL5 is mediated by Stat1 in early stages, while persistent induction of CCL5 requires activation of Stat3.

Previous reports concerning the role of CCL5 in tumors were controversial. While some studies suggest that CCL5 production may lead to a more immune suppressive TME, other evidence suggests that CCL5 in TME is in favor of tumor immunity. First, tumor expressed CCL5 positively correlates with CD8⁺ T cell infiltration in a variety of human tumors. In stage IV melanoma patients, it was found that the presence of the CCR5432 polymorphism resulted in a decreased survival following immunotherapy. Second, in experimental studies, CCL5 produced by CD4⁺ T cells induced DC infiltration into tumors, which enhanced cross priming of tumor specific CD8⁺ T cells. CCL5 was also shown to enhance NK cell infiltration into tumors. Third, CCL5 was shown to enhance glucose uptake and ATP generation in T cells. Moreover, CCL5 in CD8⁺ T cells enhanced T cell memory, while CCL5-deficient CD8⁺ T cells exhibited exhausted phenotype. In this work, using two sets of experiments, it was found that IL-27-induced CCL5 contributes to IL-27-mediated anti-tumor effect, as significantly less tumor inhibition was observed in anti-CCL5, and AAV-IL-27 treated mice. Additionally, intratumor delivery of CCL5 mRNA using lipid nanoparticles significantly inhibited tumor growth (FIG. 7).

Taken together, the current study revealed a novel function of IL-27 in inducing robust CCL5 production by T cells. In CD4⁺ T cells, CCL5 induction is Stat1-dependent, while in CD8⁺ T cells, induction of CCL5 appears to require activation of Stat3. Moreover, it was found that IL-27 induced CCL5 production enhances anti-tumor activity. These results show that IL-27-mediated anti-tumor effect is partially achieved by induction of CCL5 in T cells, particularly in CD8⁺ T cells.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### Tables

TABLE 1
qPCR primers used in this study.

| Application | Forward (5′→3′) | Reverse (5′→3′) | Note |
|---|---|---|---|
| Detect CCL5 mature | GTGCCAACCCAGA | ATGCCCATTTTCC | For RT-qPCR |
| mRNA expression | GAAGAAGTG (SEQ | CAGGACC (SEQ ID | |
| | ID NO: 7) | NO: 8) | |
| putative STAT1 | TGTCGTTGTTGTA | AGCATGTGTCTGG | −4000 bp of CCL5 |
| binding site #1 | TAGGGAGTTC | AAGGATG (SEQ ID | TSS |
| | (SEQ ID NO: 9) | NO: 10) | |
| putative STAT1 | CAAGCTGAGCTGG | ACGACTGACTAA | −4200 bp of CCL5 |
| binding site #2 | CTCTC (SEQ ID NO: | AGCACTGG (SEQ | TSS |
| | 11) | ID NO: 12) | |
| putative STAT1 | GGATCACCTTCTG | GCAACTAGAGAC | −4500 bp of CCL5 |
| binding site #3 | CTTCCTTAC (SEQ | ATGGGTGATG | TSS |
| | ID NO: 13) | (SEQ ID NO: 14) | |
| putative STAT1 | CTTCTCACCTTCA | TGTGACCCAAGC | −4700 bp of CCL5 |
| binding site #4 | CCACACAG (SEQ | ACAGTTC (SEQ ID | TSS |
| | ID NO: 15) | NO: 16) | |
| putative STAT3 | CAGGGTAGCAGA | GCAGTTAGAGGC | −200 bp of CCL5 |
| binding site #1 | GGAAGTG (SEQ ID | AGAGTCATAC | TSS |
| | NO: 17) | (SEQ ID NO: 18) | |
| putative STAT3 | CTTCTCACCTTCA | TGTGACCCAAGC | −4700 bp of CCLS |
| binding site #2 | CCACACAG (SEQ | ACAGTTC (SEQ ID | TSS |
| | ID NO: 19) | NO: 20) | |
| RNA polymerase II | TGCTACCCTGGCT | GATGCATGTGCTG | |
| | CCCTATAA (SEQ | TCTCAGAGT (SEQ | |
| | ID NO: 21) | ID NO: 22) | |

SEQUENCES

SEQ ID NO: 1 (mouse)
ATGAAGATCTCTGCAGCTGCCCTCACCATCATCCTCACTGCAGCCGCCCTCTGCA
CCCCCGCACCTGCCTCACCATATGGCTCGGACACCACTCCCTGCTGCTTTGCCTA
CCTCTCCCTCGCGCTGCCTCGTGCCCACGTCAAGGAGTATTTCTACACCAGCAGC
AAGTGCTCCAATCTTGCAGTCGTGTTTGTCACTCGAAGGAACCGCCAAGTGTGTG
CCAACCCAGAGAAGAAGTGGGTTCAAGAATACATCAACTATTTGGAGATGAGCT
AA
SEQ ID NO: 2 (Mouse)
MKISAAALTIILTAAALCTPAPASPYGSDTTPCCFAYLSLALPRAHVKEYFYTSSKCSN
LAVVFVTRRNRQVCANPEKKWVQEYINYLEMS
SEQ ID NO: 3 mRNA (mouse)
AUGAAGAUCUCUGCAGCUGCCCUCACCAUCAUCCUCACUGCAGCCGCCCUCUG
CACCCCCGCACCUGCCUCACCAUAUGGCUCGGACACCACUCCCUGCUGCUUUGC
CUACCUCUCCCUCGCGCUGCCUCGUGCCCACGUCAAGGAGUAUUUCUACACCA
GCAGCAAGUGCUCCAAUCUUGCAGUCGUGUUUGUCACUCGAAGGAACCGCCAA
GUGUGUGCCAACCCAGAGAAGAAGUGGGUUCAAGAAUACAUCAACUAUUUGG
AGAUGAGCUAA
SEQ ID NO: 4 coding sequence (human CCL5)
ATGAAGGTCTCCGCGGCAGCCCTCGCTGTCATCCTCATTGCTACTGCCCTCTGCG
CTCCTGCATCTGCCTCCCCATATTCCTCGGACACCACACCCTGCTGCTTTGCCTAC
ATTGCCCGCCCACTGCCCCGTGCCCACATCAAGGAGTATTTCTACACCAGTGGCA
AGTGCTCCAACCCAGCAGTCGTCTTTGTCACCCGAAAGAACCGCCAAGTGTGTGC
CAACCCAGAGAAGAAATGGGTTCGGGAGTACATCAACTCTTTGGAGATGAGCTA
G
SEQ ID NO: 5 Protein (human ccl5)
MKVSAAALAVILIATALCAPASASPYSSDTTPCCFAYIARPLPRAHIKEYFYTSGKCSN
PAVVFVTRKNRQVCANPEKKWVREYINSLEMS
SEQ ID NO: 6 mRNA (human ccl5)
AUGAAGGUCUCCGCGGCAGCCCUCGCUGUCAUCCUCAUUGCUACUGCCCUCUG
CGCUCCUGCAUCUGCCUCCCCAUAUUCCUCGGACACCACACCCUGCUGCUUUG
CCUACAUUGCCCGCCCACUGCCCCGUGCCCACAUCAAGGAGUAUUUCUACACC
AGUGGCAAGUGCUCCAACCCAGCAGUCGUCUUUGUCACCCGAAAGAACCGCCA
AGUGUGUGCCAACCCAGAGAAGAAAUGGGUUCGGGAGUACAUCAACUCUUUG
GAGAUGAGCUAG

1. A composition comprising: a lipid-like nanoparticle encapsulating a CCL5 mRNA.

2. The composition of claim 1, wherein the CCL5 mRNA is encoded by SEQ ID NO: 1 or SEQ ID NO: 4.

3. The composition of claim 1, wherein the lipid-like nanoparticle comprises a compound of Formula I: 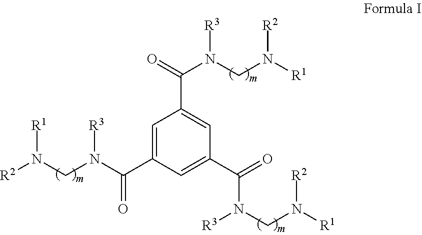
and salts thereof; wherein
each R¹ is independently hydrogen or substituted or unsubstituted alkyl;
each R² is independently substituted or unsubstituted alkyl;
each R³ is independently hydrogen or substituted or unsubstituted alkyl; and
each m is independently 1, 2, 3, 4, 5, 6, 7, or 8.

4. The composition of claim 1, wherein the lipid-like nanoparticle comprises a compound of Formula II: 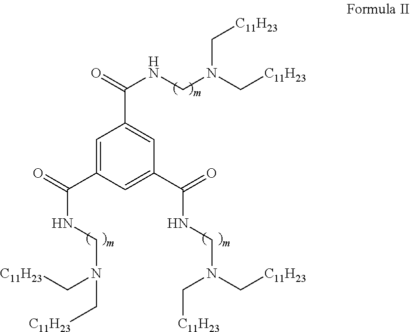
and salts thereof, wherein m=3.

5. The composition of claim 1, wherein the lipid-like nanoparticle further comprises:
a non-cationic lipid;
a polyethylene glycol-lipid; and
a sterol.

6. The composition of claim 5, wherein the non-cationic lipid is a phosphatidylethanolamine lipid.

7. The composition of claim 6, wherein the phosphatidylethanolamine lipid is selected from 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), or combinations thereof.

8. The composition of claim 5, wherein the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs.

9. The composition of claim 5, wherein the sterol is selected from cholesterol, campesterol, ergosterol, or sitosterol.

10. A method of treating a cancer in a subject comprising administering to the subject a composition comprising: a lipid-like nanoparticle encapsulating a CCL5 mRNA.

11. The method of claim 10, wherein the CCL5 mRNA is encoded by SEQ ID NO: 1 or SEQ ID NO: 4.

12. The method of claim 10, wherein the subject is further administered an anticancer agent.

13. The method of claim 12, wherein the anticancer agent is selected from a PD-L1 antibody, a PD-1 antibody, or a CTLA-4 antibody.

14. The method of claim 10, wherein the lipid-like nanoparticle comprises:
a compound of Formula I: 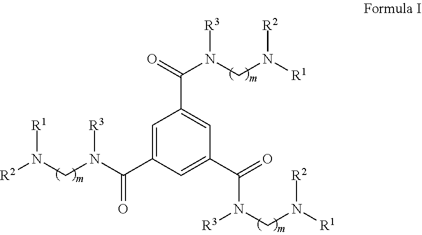
and salts thereof; wherein
each R¹ is independently hydrogen or substituted or unsubstituted alkyl;
each R² is independently substituted or unsubstituted alkyl;
each R³ is independently hydrogen or substituted or unsubstituted alkyl; and
each m is independently 1, 2, 3, 4, 5, 6, 7, or 8.

15. The method of claim 10, wherein the lipid-like nanoparticle comprises a compound of Formula II: 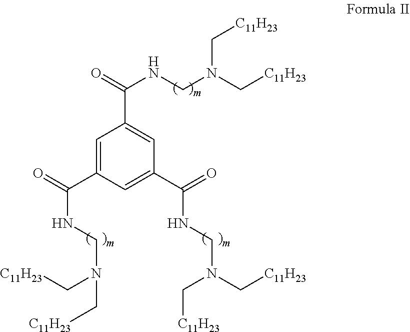
and salts thereof, wherein m=3.

16. The method of claim 10, wherein the lipid-like nanoparticle further comprises:
a non-cationic lipid;
a polyethylene glycol-lipid; and
a sterol.

17. The method of claim 16, wherein the non-cationic lipid is a phosphatidylethanolamine lipid.

18. The method of claim 17, wherein the phosphatidylethanolamine lipid is selected from 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), or combinations thereof.

19. The method of claim 16, wherein the polyethylene glycol-lipid is selected from 1,2-dimyristoyl-sn-glycerol, methoxypolyethylene glycol (DMG-PEG), DLPE-PEGs, DMPE-PEGs, DPPC-PEGs, and DSPE-PEGs.

20. The method of claim 16, wherein the sterol is selected from cholesterol, campesterol, ergosterol, or sitosterol.

21. The method of claim 10, wherein the cancer is a melanoma.
